(19) United States
(12) Patent Application Publication
(10) Pub. No.: US 2015/0174232 A1
(43) Pub. Date: Jun. 25, 2015

(54) COMPOSITION AND USES THEREOF

(71) Applicant: University of Southampton, Southampton (GB)

(72) Inventors: Myron Christodoulides, Southampton (GB); John Edwards Heckels, Southampton (GB)

(21) Appl. No.: 14/409,724

(22) PCT Filed: Jun. 20, 2013
(86) PCT No.: PCT/GB2013/051621

§ 371 Date: Dec. 19, 2014

(30) Foreign Application Priority Data
Jun. 22, 2012 (GB) 1211154.8

(51) Int. Cl. A61K 39/095

(52) U.S. Cl. CPC A61K 39/095; A61K 2039/55555

(57) ABSTRACT

An immunogenic composition comprising the Nm-ACP protein or an immunogenic part thereof or an analogue thereof, wherein the composition is capable of eliciting an immune response when administered to a human or non-human animal.

The present invention relates to immunogenic compositions for use in eliciting an immune response to a pathogenic organism, and in particular, to immunogenic compositions capable of eliciting protective immune responses.

Infections caused by Neisseria meningitidis (meningococcus) are significant causes of mortality and morbidity worldwide. Despite the success of capsular polysaccharide-protein conjugate vaccines against meningococcal serogroup A, C, Y and W-135 introduced into the routine immunisation schedules of developed countries (Borrow and Miller 2006 Expert Review of Vaccines 5: 851-857 and MD, et al. (2008), JAMA 299, 173-184), no such vaccines exist for serogroup B infection. The conjugate strategy that has been successful for both serogroups A, C, Y and W-135 capsule is unlikely to be effective for serogroup B due to structural similarities between the B capsule and the human foetal brain NCAM, thus raising the possibility of inducing auto-immune responses. As a result, serogroup B vaccine development has focused on the use of isolated sub-capsular outer membrane (OM) proteins for epidemic control, but these are complex and protection is PorA sero-subtype specific (Hoist et al 2009 Vaccine 27: B3-B12). Current licensed MenB vaccines have been prepared by detergent treatment of outer membranes (OM) to form lipopolysaccharide (LPS)-depleted OMV (vesicle) vaccines, which provide only strain-specific protection. Many individual OM and secreted proteins have been tested for their ability to induce serum bactericidal antibodies, the generally accepted correlate of protection against meningococcal infection. A multi-component vaccine (4CMenB) that includes factor H binding protein (fHbp), Neisserial heparin binding antigen (NHBA) and a minor adhesin (NadA), in complex with OMV from a MenB outbreak in New Zealand (PorA 1.4) has undergone phase III trials. Trials of another vaccine (LP2086) containing two subfamilies of fHbp have also reached phase II. However, many OM antigens are hyper-variable and a goal for effective vaccine development is to identify those antigens that are more conserved and capable of inducing cross-protective antibody responses.

Thus, the goal is to identify antigens present in the OM that are conserved and capable of inducing cross-protective antibody responses. Recently, reverse vaccinology has led to the development of a pentavalent recombinant protein vaccine, 5CMBV, which has been used in clinical trials with success when administered with OM (Giuliani et al 2006 Proc.Nat.Acad.Sci. 103: 10834-10839). In addition, many proteins present in the OM, such as the major porins PorA and PorB, the opacity protein Opc, fHBP and others have also been prepared as recombinant proteins and investigated as experimental vaccines. However, many of these antigens are variable and unsuitable as vaccine candidates.

An aim of the present invention is provide one or more compositions which can be used to elicit a protective immune response against Neisseria meningitidis (N. meningitidis), and serogroup B in particular

The present invention relates to novel compositions, and in particular, to novel immunogenic compositions, comprising the N. meningitidis-outer membrane protein Adhesin Complex Protein (Nm-ACP), and to the use of these compositions to elicit an immune response against N. meningitidis.

According to a first aspect, the present invention provides an immunogenic composition comprising the Nm-ACP protein or an immunogenic part thereof or an analogue thereof, wherein the composition is capable of eliciting an immune response when administered to a human or non-human animal.

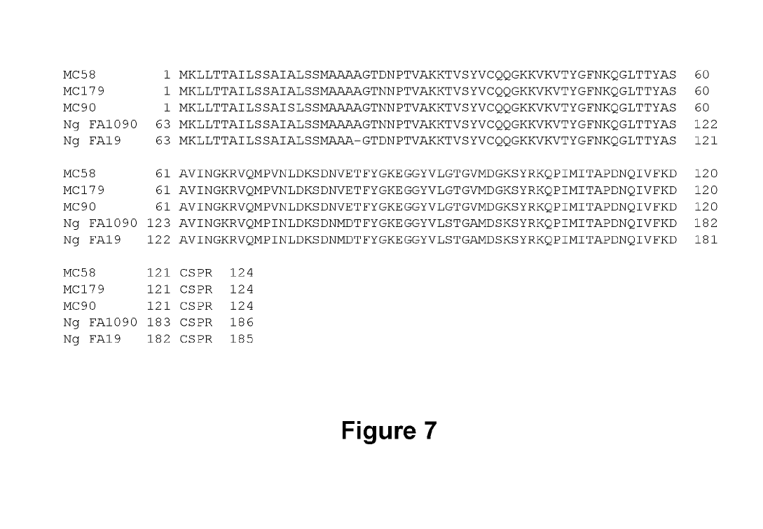

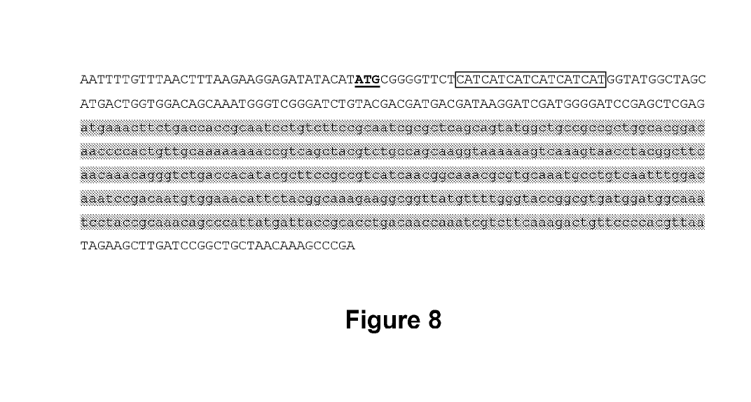

The Nm-ACP protein is present in the outer membrane of N. meningitidis. The Nm-ACP protein is encoded by the NMB2095 gene (the acp gene) in the MenB genome the sequence of which can be accessed from the NCBI website (http://www.ncbi.nlm.nih.gov/) using accession number NMB2095 in the MenB genome. The acp gene sequence is also shown in FIG. 8 with reference to strain MC58. The Nm-ACP protein is a 124 amino acid protein as shown in FIG. 7 with reference to strain MC58 (Sequence ID No: 1) and has a molecular weight of 13.3 kDa.

As used in the present application, an “analogue” can include a variant in which one or more residues are added, deleted, inserted or substituted, while having no material effect on the function of the protein. That is, an analogue in accordance with one aspect of the present invention should be capable of inducing an antibody or T-cell response to the Nm-ACP protein. A residue (or residues) may be added or deleted from either end of the protein, deleted from within the protein, inserted within the protein, or substituted for one or more of the residues within the protein. As would be understood by a person of ordinary skill in art, one or more protein residues may be added, deleted, inserted or substituted while still maintaining the function of the protein. For example, as many as five or more residues may be added to or removed from either end of a protein, or inserted into a protein, and be considered a protein analogue within the context of the present invention. In a further example, a conservative substitution of one or more residues within a protein may result in a protein analogue. As would be well understood to the skilled artisan, a conservative substitution includes a substitution of one amino acid residue with another amino acid residue having one or more similar chemical properties, such as polarity, charge, hydrophobicity, or aromaticity, for example.

The Nm-ACP protein, or an immunogenic part thereof, or an analogue thereof, may further comprise additional sequence, for example, for use in purification of the protein. For example, if the protein is a recombinant protein the protein may include a his-tag sequence for use in protein purification. The protein used in the method of the invention may have the sequence of Sequence ID No: 1, 2, 3, 4 or 5 (FIG. 7).

As used in the present application, an “immunogenic part” may include any part of the Nm-ACP protein that can be used to elicit a protective immune response when administered to a human or non-human animal. The composition may also comprise an analogue of an immunogenic part of the Nm-ACP protein.

Reference herein to the Nm-ACP protein is intended to refer to the Nm-ACP protein of Sequence ID No: 1, 2, 3, 4 or 5 or to a protein with 50%, 60%, 70%, 80%, 90%, 95% or more, preferably 80% or more, preferably 85% or more, preferably 90% or more, preferably 95% or more, sequence homology with the Nm-ACP protein of Sequence ID No: 1, 2, 3, 4 or 5.

Reference to percentage homology relates to the percent identity between two aligned sequences. The percent identity refers to the residues in two proteins which are the same, when the protein sequences are aligned for maximum correspondence and when inversions and translocations are accounted for. Preferably the percent identity ignores any conservative differences between the aligned sequences which do not affect function. The percent identity between aligned sequences can be established by using well-established tools (such as the BLAST algorithm—Basic Local Alignment Search Tool; Altschul et al., (1990) J Mol Biol. 215:403-10

Variations in percent identity may be due, for example, to amino acid substitutions, insertions or deletions. Amino acid substitutions may be conservative in nature.

The immunogenic composition of the invention may comprise the Nm-ACP of Sequence ID No 1, 2, 3, 4 or 5 or an analogue thereof, wherein the Nm-ACP or analogue thereof is capable of eliciting an immune response when administered to a human or non-human animal.

The immunogenic composition of the invention may comprise the Nm-ACP protein having a sequence of Sequence ID No 1, 2, 3, 4 or 5 wherein the Nm-ACP protein is capable of eliciting an immune response when administered to a human or non-human animal. The immunogenic composition of the invention may comprise a fragment or variant of the Nm-ACP protein of SEQ ID No. 1 wherein the fragment or variant of the Nm-ACP protein is capable of eliciting an immune response when administered to a human or non-human animal. The fragment or variant of the Nm-ACP protein may be any fragment or variant that is capable of eliciting an immune response. The fragment or variant of the Nm-ACP protein may have more than 40%, more than 50%, more than 60%, more than 70%, more than 80%, more than 90%, more than 95%, more than 98% or more than 99% sequence identity to SEQ ID No. 1, 2, 3, 4 or 5.

The immunogenic composition of the invention may comprise an Nm-ACP protein encoded by the sequence of SEQ ID No. 7, an Nm-ACP fusion protein encoded by the sequence of SEQ ID No. 8 or an Nm-ACP protein encoded by the sequence of SEQ ID No. 8 but having the linker and tag cleaved off, for example using a suitable restriction endo-nuclease, wherein the Nm-ACP protein is capable of eliciting an immune response when administered to a human or non-human animal. The immunogenic composition of the invention may comprise a fragment or variant of an Nm-ACP protein encoded by the sequence of SEQ ID No. 7, an Nm-ACP fusion protein encoded by the sequence of SEQ ID No. 8 or an Nm-ACP protein encoded by the sequence of SEQ ID No. 8 but having the linker and tag cleaved off, wherein the fragment or variant of the Nm-ACP protein is capable of eliciting an immune response when administered to a human or non-human animal. The fragment or variant of the Nm-ACP protein may be any fragment or variant that is capable of eliciting an immune response. The fragment or variant of the Nm-ACP protein may have more than 40%, more than 50%, more than 60%, more than 70%, more than 80%, more than 90%, more than 95%, more than 98% or more than 99% sequence identity to an Nm-ACP protein encoded by the sequence of SEQ ID No. 7, an Nm-ACP fusion protein encoded by the sequence of SEQ ID No. 8 or an Nm-ACP protein encoded by the sequence of SEQ ID No. 8 but having the linker and tag cleaved off.

The Nm-ACP protein may be an Nm-ACP fusion protein where the Nm-ACP protein is fused to another polypeptide or protein, for example a linker protein, a tag for use in purification of proteins or a soluble protein.

The compositions of the present invention are particularly suitable for preparing immunogenic compositions, such as vaccines, for use in the prevention of N. meningitidis infection. However, it will be appreciated that the compositions may be used in the prevention of infection by other related pathogens, or pathogens with a similar Nm-ACP protein. Such pathogens include Neisseria gonorrhoeae.

The immune response elicited by a composition of the invention may be effective against N. meningitidis serogroup B strains, the immune response may also be effective against N. meningitidis serogroup A strains and/or N. meningitidis serogroup C strains and/or N. meningitidis serogroup W135 strains and/or N. meningitidis serogroup Y strains. The immune response elicited may also or alternatively be effective against Neisseria gonorrhoeae (N. gonorrhoeae).

The immune response elicited by the composition of the invention may affect the ability of N. meningitidis and/or N. gonorrhoeae to infect an immunised animal. Preferably the ability of N. meningitidis and/or N. gonorrhoeae to infect a human immunised with the composition of the invention is impeded or prevented. This may be achieved in a number ways. The immune response elicited may recognise and destroy N. meningitidis and/or N. gonorrhoeae. Alternatively, or additionally, the immune response elicited may impede or prevent the replication of N. meningitidis and/or N. gonorrhoeae. Alternatively, or additionally, the immune response elicited may impede or prevent N. meningitidis and/or N. gonorrhoeae causing disease in the human or non-human animal. Preferably the immune response elicited is directed to at least N. meningitidis serogroup B.

The Nm-ACP protein may be recovered from N. meningitidis and/or it may be produced recombinantly and/or it may a synthetic product, for example produced by in vitro peptide synthesis or in vitro translation

The composition of the invention may also comprise a further one or more antigens, in addition to the Nm-ACP protein or an immunogenic part thereof or an analogue thereof. The further antigens may also be derived from N. meningitidis, and may be capable of eliciting an immune response directed to N. meningitidis.

The composition may be used to elicit/produce a protective immune response when administered to a subject. The protective immune response may cause N. meningitidis to be killed upon infecting the subject, or it may prevent or inhibit N. meningitidis from replicating and/or from causing disease.

The composition may be used as a prophylactic or a therapeutic vaccine directed to N. meningitidis, and in particular N. meningitidis serogroup B. The composition may also or alternatively be used as a prophylactic or a therapeutic vaccine directed to N. gonorrhoeae.

According to a further aspect, the invention provides a pharmaceutical composition comprising the Nm-ACP protein or an immunogenic part thereof or an analogue thereof and a pharmaceutically acceptable carrier or excipient.

The pharmaceutical composition may comprise a composition according to the first aspect of the invention.

The pharmaceutical composition may be capable of producing a protective immune response to N. meningitidis.

The phrase “producing a protective immune response” as used herein means that the composition is capable of generating a protective response in a host organism, such as a human or a non-human mammal, to whom it is administered. Preferably a protective immune response protects against subsequent infection by N. meningitides. The protective immune response may eliminate or reduce the level of infection by reducing replication of N. meningitidis, or by affecting the mode of action of N. meningitidis to reduce disease. Preferably the protective response is directed to at least N. meningitidis serogroup B.

Suitable acceptable excipients and carriers will be well known to those skilled in the art. These may include solid or liquid carriers. Suitable liquid carriers include water and saline. The proteins of the composition may be formulated into an emulsion or they may be formulated into biodegradable microspheres or liposomes.

The composition of the invention may be incorporated into liposomes or detergent micelles for administration.

The composition of the invention may comprise immunogenic Nm-ACP protein in saline solution.

Preferably, the Nm-ACP protein or immunogenic part thereof or analogue thereof in the composition of the invention is folded into its native configuration.

The composition may further comprise an adjuvant, wherein an adjuvant enhances the protective efficacy of the composition. Suitable adjuvants will be well known to those skilled in the art, and may include emulsifiers, muramyl dipeptides, avridine, aqueous adjuvants such as aluminium hydroxide, chitosan-based adjuvants, monophosphoryl Lipid A and any of the various saponins, oils, and other substances known in the art, such as Amphigen, LPS, bacterial cell wall extracts, bacterial DNA, CpG sequences, synthetic oligonucleotides and combinations thereof. Other suitable adjuvants can be formed with an oil component, such as a single oil, a mixture of oils, a water-in-oil emulsion, or an oil-in-water emulsion. The oil may be a mineral oil, a vegetable oil, or an animal oil. Mineral oils are liquid hydrocarbons obtained from petrolatum via a distillation technique, and are also referred to in the art as liquid paraffin, liquid petrolatum, or white mineral oil. Suitable animal oils include, for example, cod liver oil, halibut oil, menhaden oil, orange oil and shark liver oil, all of which are available commercially. Suitable vegetable oils, include, for example, canola oil, almond oil, cottonseed oil, corn oil, olive oil, peanut oil, safflower oil, sesame oil, soybean oil, and the like. Freund's Complete Adjuvant (FCA) and Freund's Incomplete Adjuvant (FIA) are two common adjuvants that are commonly used in vaccine preparations, and are also suitable for use in the present invention. Both FCA and FIA are water-in-mineral oil emulsions; however, FCA also contains a killed Mycobacterium sp.

The adjuvant may be used in a composition of the invention is aluminium hydroxide and/or monophosphoryl Lipid A.

The adjuvant may be used in a composition of the invention is monophosphoryl Lipid A.

In one embodiment no adjuvant is included in a composition of the invention.

The composition may also comprise polymers or other agents to control the consistency of the composition, and/or to control the release of the protein from the composition.

The composition may also comprise other agents such as diluents, which may include water, saline, glycerol or other suitable alcohols etc; wetting or emulsifying agents; buffering agents; thickening agents for example cellulose or cellulose derivatives; preservatives; detergents; antimicrobial agents; and the like.

The active ingredients in the composition may be greater than 50% pure, usually greater than 80% pure, often greater than 90% pure and more preferably greater than 95%, 98% or 99% pure. With active ingredients approaching 100% pure, for example about 99.5% pure or about 99.9% pure, being used most often.

The composition of the present invention may be used as vaccine against infections caused by N. meningitidis, in particular serogroup B and possibly also serogroup A and/or serogroup C and/or serogroup W135 and/or serogroup Y and/or N. gonorrhoeae. The composition may be used as a vaccine directed to N. meningitidis or other invasive meningococcal diseases including septicaemia or septic shock, and/or as a vaccine directed to N. gonorrhoeae. The vaccine may be administered prophylactically to those at risk of exposure to N. meningitidis and/or N. gonorrhoeae, and/or therapeutically to persons who have already been exposed to N. meningitidis and/or N. gonorrhoeae.

Preferably, if the composition is used as a vaccine, the composition comprises an immunologically effective amount of antigen wherein the composition comprises the Nm-ACP protein or an immunogenic part thereof or an analogue thereof. An “immunologically effective amount” of an antigen is an amount that when administered to an individual, either in a single dose or in a series of doses, is effective for treatment or prevention of infection by N. meningitidis and/or N. gonorrhoeae. This amount will vary depending upon the health and physical condition of the individual to be treated and on the antigen. Determination of an effective amount of an immunogenic or vaccine composition for administration to an organism is well within the capabilities of those skilled in the art.

A composition according to the invention may be for oral, systemic, parenteral, topical, mucosal, intramuscular, intravenous, intraperitoneal, intradermal, subcutaneous, intranasal, intravaginal, intrarectal, transdermal, sublingual, inhalation or aerosol administration.

The composition may be arranged to be administered as a single dose or as part of a multiple dose schedule. Multiple doses may be administered as a primary immunisation followed by one or more booster immunisations. Suitable timings between priming and boosting immunisations can be routinely determined.

A composition according to the invention may be used in isolation, or it may be combined with one or more other immunogenic or vaccine compositions, and/or with one or more other therapeutic regimes.

Compositions of the invention may be able to induce serum bactericidal antibody responses and elicit antibodies which mediate opsonphagocytosis after being administered to a subject. These responses are conveniently measured in mice and the results are a standard indicator of vaccine efficacy.

The compositions of the invention may also, or alternatively, be able to elicit an immune response which neutralises bacterial proteins or other molecules, thereby preventing them from having their normal function and preventing or reducing disease progression without necessarily destroying the pathogenic organism/bacteria, in this case to N. meningitidis and/or N. gonorrhoeae.

According to a further aspect, the present invention provides the use of the Nm-ACP protein or an immunogenic part thereof or an analogue thereof in the preparation of a medicament for eliciting an immune response. The medicament may be used for the prophylactic or therapeutic vaccination of subjects against N. meningitidis and/or N. gonorrhoeae. The medicament may be a prophylactic or a therapeutic vaccine. The vaccine may be for meningitis, septicaemia and/or septic shock caused by N. meningitidis.

According to a yet further aspect, the invention provides a composition comprising the Nm-ACP protein or an immunogenic part thereof or an analogue thereof for use in generating an immune response to N. meningitidis and/or N. gonorrhoeae. The immune response may be prophylactic or therapeutic. The composition may be for use as a vaccine.

According a still further aspect, the present invention provides a method of protecting a human or non-human animal from the effects of infection by N. meningitidis and/or N. gonorrhoeae comprising administering to the human or non-human animal a composition according to any other aspect of the invention. The composition may be a vaccine.

According to another aspect, the invention provides a method for raising an immune response in a human or non-human animal comprising administering a pharmaceutical composition according to the invention to the human or non-human animal. The immune response is preferably protective. The method may raise a booster response in a patient that has already been primed. The immune response may be prophylactic or therapeutic.

One way to check the efficacy of a therapeutic treatment comprising administration of a composition according to the invention involves monitoring for N. meningitidis and/or N. gonorrhoeae infection after administration of the composition. One way to check the efficacy of a prophylactic treatment comprising administration of a composition according to the invention involves monitoring immune responses to N. meningitidis and/or N. gonorrhoeae after administration of the composition.

According to another aspect, the invention provides the use of the Nm-ACP protein or an immunogenic part thereof or an analogue thereof in the preparation of a medicament for use in the immunisation of human or non-human mammals against infection by N. meningitidis and/or N. gonorrhoeae.

According to a further aspect the invention provides a kit for use in inducing an immune response in an organism, comprising an immunogenic or vaccine composition according to the invention and instructions relating to administration.

In addition to their potential use as vaccines, compositions according to the invention may be useful as diagnostic reagents and as a measure of the immune competence of a vaccine.

In a composition of the invention the antigenic component may consist only of, or substantially only of, an Nm-ACP protein or an immunogenic part thereof or an analogue thereof, as described herein.

In a composition of the invention the Nm-ACP protein or an immunogenic part thereof or an analogue thereof, as described herein, may be used in a purified, or substantially purified form, preferably the Nm-ACP protein or an immunogenic part thereof or an analogue thereof is not provided as part of a membrane preparation.

While all the statements of invention and preferable features discussed above refer to N. meningitidis, the skilled man will appreciate that they could equally apply to N. gonorrhoeae.

It will be appreciated that optional features applicable to one aspect of the invention can be used in any combination, and in any number. Moreover, they can also be used with any of the other aspects of the invention in any combination and in any number. This includes, but is not limited to, the dependent claims from any claim being used as dependent claims for any other claim in the claims of this application.

Preferred embodiments of the present invention will now be described, merely by way of example, with reference to the following figures and examples.

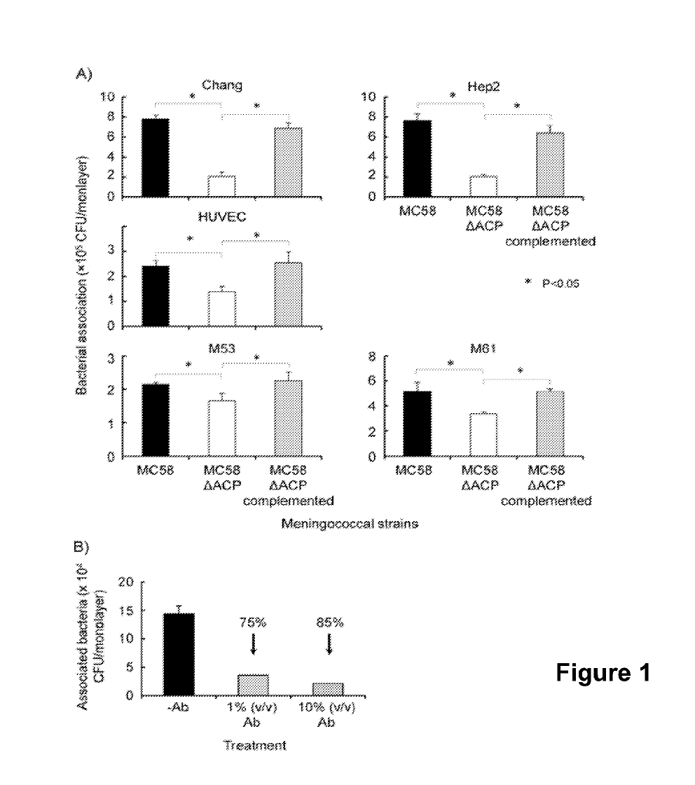

FIG. 1—A) shows the results of experiments that show that Nm-ACP mediates adhesion of meningococci to human cells. Chang, Hep2, HUVEC and meningioma (M53, M61) monolayers were infected with equal MOI of MC58, MC58ΔACP and the MC58ΔACP complemented strains, ranging from 100-5000 for individual experiments. Representative experiments for each cell line are shown, as suggested by Virji et al ((1995) Mol Microbiol. 18, 741-754). Experiments were repeated 5-6 times for each cell line and the patterns of association of the different bacterial strains were reproducible between experiments, regardless of any quantitative variations between experiments. The columns denote the mean associated bacterial numbers and the error bars show the standard deviations from triplicate wells.

B) shows results from an experiment showing that anti-rACP serum inhibits bacterial association to epithelial cells. Chang epithelial cells were infected with MC58 (ACP+) in the presence and absence of decomplemented rabbit anti-rACP serum and bacterial association measured after 3 h. The data are representative of n=3 experiments using an inoculum 5×10⁴ CFU/monolayer. Pilot experiments showed that with different doses (10⁴-10⁸ CFU/monolayer) of MC58 in the presence of 10% (v/v) anti-rACP serum, the binding of antibody to ACP reached saturation when the inoculum was >10⁷ CFU/monolayer. The columns represent the mean associated bacterial numbers and the error bars show the standard deviations from triplicate wells.

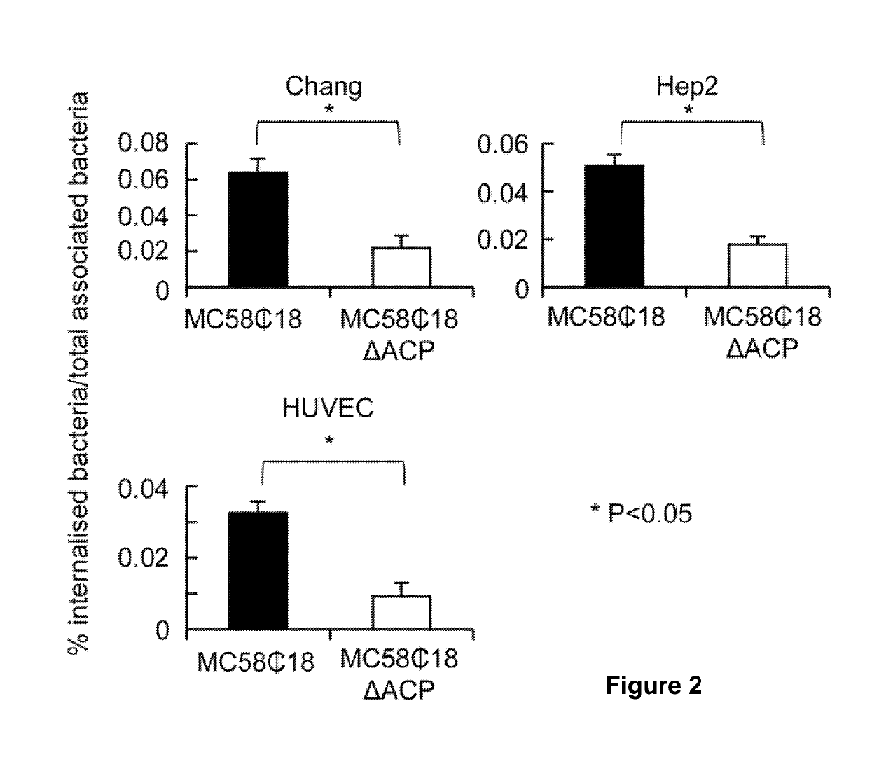

FIG. 2—shows the results of an experiment showing that Nm-ACP mediates invasion of human cells by meningococci. Chang, Hep2 and HUVEC monolayers were challenged with MC58¢18 and MC58¢18ΔACP, ranging from MOI of 750-6000 for individual experiments, and the numbers of internalized bacteria estimated using the gentamicin assay. Representative experiments for each cell line are shown, as suggested by Virji et al ((1995) Mol Microbiol. 18, 741-754). Experiments were repeated 3-5 times for each cell line and the differences in invasion between the strains were reproducible between experiments, regardless of any quantitative variations between experiments. The columns represent the mean percentage of internalised bacteria and the error bars the standard deviations from triplicate wells.

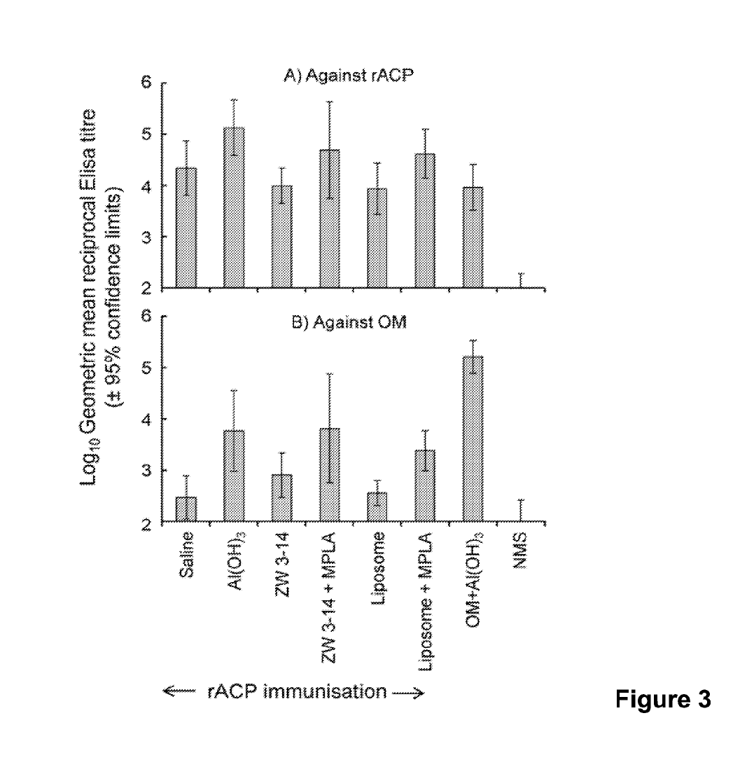

FIG. 3—shows ELISA reactivity of antisera raised against different rACP formulations. Anti-rACP sera were reacted against A) rACP and B) MC58 OM. The columns represent the geometric mean of reciprocal ELISA titres (n=4-5 animals per group) and the error bars show the 95% confidence limits. No reactivity against either rACP or homologous OM was observed with sera from sham immunised animals.

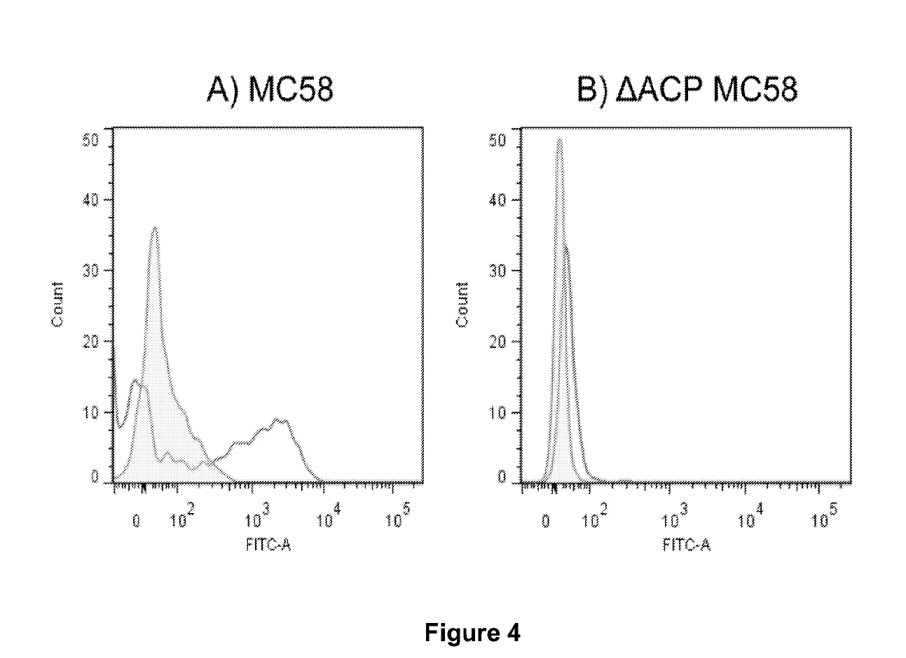

FIG. 4—FACS analysis demonstrates expression of Nm-ACP on the surface of meningococci. Shaded area shows the reactivity with pre-immune rabbit sera. Non-shaded area shows the reactivity of rabbit anti rACP sera to A) MC58 (ACP+) and B) MC58ΔACP (ACP−).

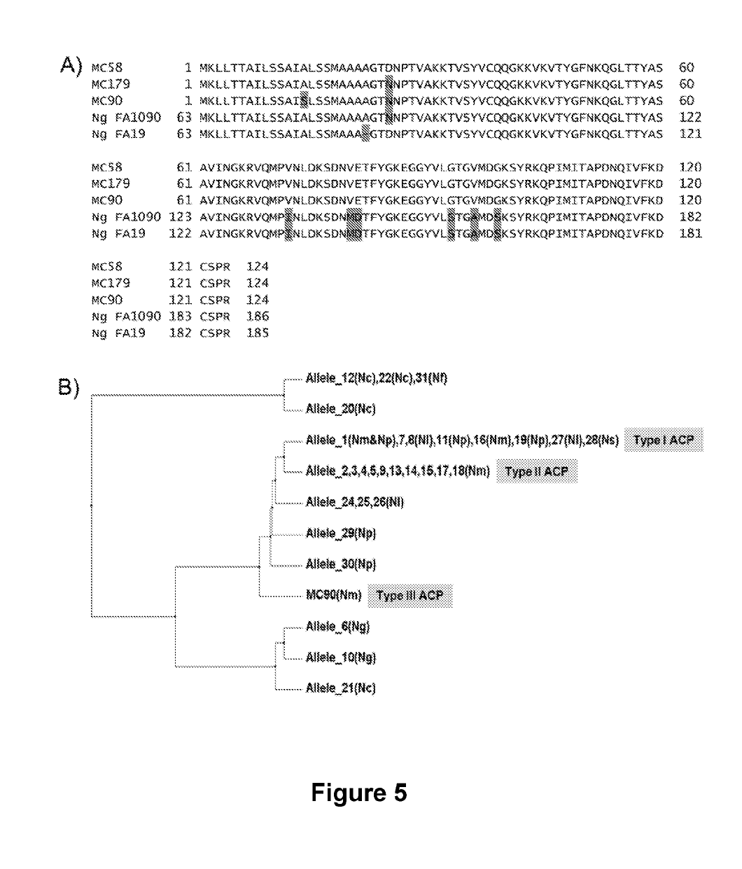

FIG. 5—A) shows an alignment of ACP amino acid sequences from MC58, the surveyed strains in the inventors collection and from gonococci. Grey shading denotes the amino acid differences compared with MC58. B) shows a dendrogram showing the clustering of 11 ACP proteins and the relationships between type I, II and III ACP in Neisseria spp.

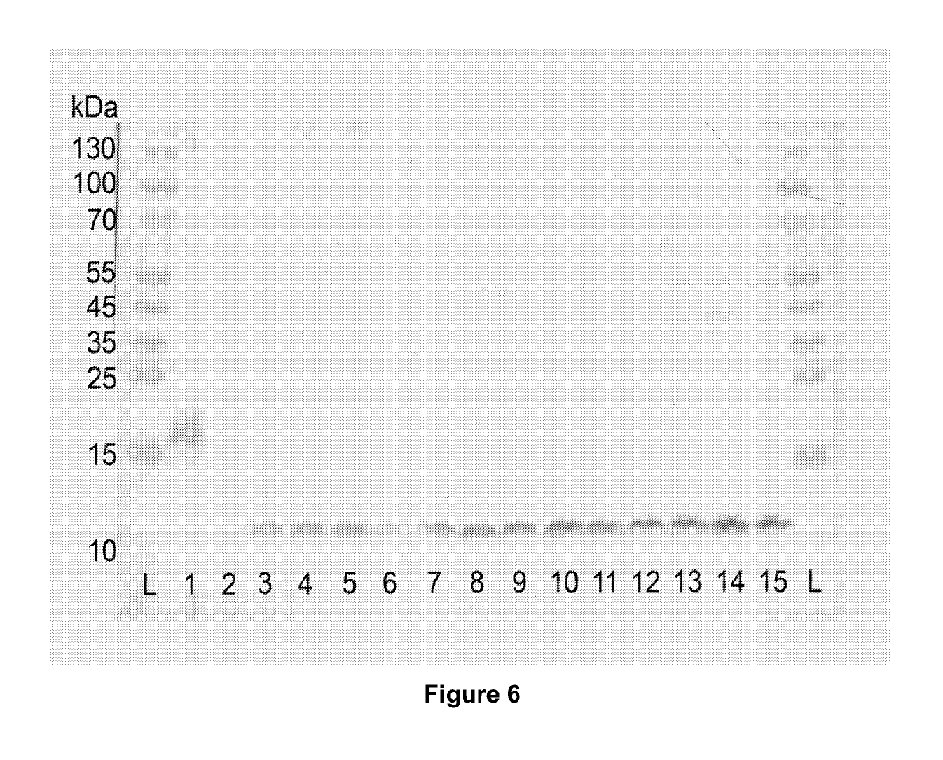

FIG. 6—shows western blot reactivity of rabbit antisera ( 1/400 dilution) to Nm-ACP in lysates of meningococcal strains. Lane M; protein markers. Lane 1; rACP (0.1 μg). Lane 2 MC58ΔACP (20 μg). Lane 3, 4: MC58, MC168 (type 1, 20 μg). Lane 5-14: L2470, MC54, MC161, MC162, MC172, MC173, MC174, MC179, MC180, MENC11 (type II, 20 μg). Lane 15: MC90 (type III, 20 μg).

FIG. 7—details the sequence of the Nm-ACP protein isolated from N. meningitidis strain MC58 (SEQ ID No: 1), N. meningitidis strain MC179 (SEQ ID No: 2), N. meningitidis strain MC90 (SEQ ID No: 3), Ng FA1090 (SEQ ID No: 4), and Ng FA19 (SEQ ID No: 5).

FIG. 8—details the nucleotide sequence encoding of the Nm-ACP protein isolated from N. meningitidis strain MC58. The acp gene (375 bp) sequence (SEQ ID No. 6) is highlighted in grey. The whole sequence shown in FIG. 8 (SEQ ID No. 7) is the sequence that was cloned into an expression vector. The sequence highlighted in grey is the Nm-ACP sequence from strain MC58. The underlined ATG in SEQ ID No. 7 is the start codon and the sequence in a box encodes a poly-histidine tag. Between the poly-histidine tag and the sequence highlighted in grey is a linker sequence. The whole of SEQ ID No. 7 was inserted into an expression vector and an Nm-ACP-fusion protein of molecular weight approximately 17 kDa was produced. The linker and histidine tag may be cleaved off the fusion protein before it is used in an immunogenic composition or the Nm-ACP-fusion protein may be used as an immunogenic composition without cleaving off the linker sequence and tag.

### MATERIALS AND METHODS

Bacteria and Growth Conditions.

Neisseria meningitidis strain MC58 (B:15:P1.7, 16b) and other meningococcal strains of different serogroup, serotype and serosubtype, originally isolated from patients or colonized individuals, have been described previously (Hung M C et al. (2011), Infect. Immun. 79, 3784-3791). A non-capsular mutant strain MC58¢18 (PilE-, Opa-, Opc-) was kindly provided by Professor M. Virji (University of Bristol, UK) (McNeil G, Virji M (1997) Microb. Pathog. 22, 295-304). Meningococci were grown on GC-agar plates incubated at 37° C. in an atmosphere containing 5% (v/v) CO₂ and OM were prepared by extraction of whole cells by lithium acetate as previously described (Christodoulides M, (1998) Microbiology 144 (Pt 11), 3027-3037). E. coli strain DH5α and BL21 (DE3)pLysS (Invitrogen, UK) were used for cloning and protein expression and were grown on Luria Bertani (LB) agar and in LB broth.

Cloning and Expression of Acp Gene in E. coli

Genomic DNA of MC58 was extracted as described previously (Hung M C, (2011) Infect. Immun. 79, 3784-3791) and used as the PCR template. The acp gene (375 bp) sequence was accessed from NCBI website and amplified using the forward primer (NMB2095F: 5′-ggctatctcgagatgaaacttctgaccaccgc-3′ SEQ ID No. 8), the reverse primer (NMB2095R: 5′-ggctataagcttctattaacgtggggaacagtctt-3′ SEQ ID No. 9) and 2× Phusion™ PCR master mix (Finnzymes, UK) under the following PCR conditions: initial denaturation (98° C., 30 s) and then 30 cycles of denaturation (98° C., 10 s), annealing (65° C., 30 s), and extension (72° C., 10 s) and a final extension at 72° C. for 5 min. The ctcgag and aagctt represent the restriction sites for XhoI and HindIII enzymes respectively. The method for gene cloning into pRSETA system was described previously (Hung M C, (2011) Infect. Immun. 79, 3784-3791). Next, recombinant plasmids carrying the sequence-proved acp gene were transformed into E. coli BL21 (DE3)pLysS for protein expression, which was induced by addition of IPTG to a final concentration of 1 mM.

Purification of rACP

The insoluble rACP was purified using nickel-nitrilotriacetic acid metal-affinity chromatography under denaturing conditions as described in the QIAexpressionist system manual (Qiagen, UK). The bound protein was eluted using 100 mM NaH₂PO₄, 10 mM Tris buffer containing 6 M GuHCl, pH 4.5 and then precipitated by adding trichloroacetic acid (TCA, BDH, UK) to 10% (v/v final concentration). Samples obtained during the purification procedure were analysed using 10-25% (w/v) gradient SDS-PAGE (Hung M C, (2011) Infect. Immun. 79, 3784-3791) or tricine-SDS-PAGE (Schagger H (2006) Tricine-SDS-PAGE Nat. Protoc. 1, 16-22) and protein concentration was determined using the BCA assay (Pierce®, Thermo-Scientific, UK) following the manufacturer's instructions. The presence of any contaminating LPS was detected with the Limulus Amebocyte Lysate (LAL) assay, following the manufacturer's instructions (Lonza, UK).

### Immunisation of Animals

BALB/C mice (H-2d) and New Zealand white rabbits were housed under standard conditions of temperature and humidity. Groups of five mice of approximate equal size and weight (6/7 weeks of age) were immunized intra-peritoneally with rACP-saline, rACP-Al(OH)₃, rACP-liposomes, rACP+MPLA-liposomes, rACP-ZW 3-14 micelles or rACP+MPLA-ZW 3-14 micelles. The methods for preparing liposomes and other adjuvant mixtures have been described previously (Hung M C, (2011) Infect. Immun. 79, 3784-3791). The immunization schedule was three doses of rACP (20 μg/mouse) on day 0, 14 and 28. Groups of five mice were also injected with the same preparations without rACP (sham controls), one group was injected with MC58 OM (20 μg/mouse) adsorbed to Al(OH)3 and another group was kept for normal serum. Mice were terminally bled by cardiac puncture under anaesthesia on day 42.

Rabbits (n=2) were immunized subcutaneously with rACP (20 μg/dose) emulsified in Freund's Complete Adjuvant for the primary injection and Freund's Incomplete Adjuvant for a subsequent three injections at 14 day intervals. Rabbits were terminally bled from the middle ear vein and by cardiac puncture under anaesthesia, 14 days after the last dose.

All sera were stored at −20° C. until required. This study complied with the animal experimentation guidelines of the Home Office and the authors' institution and no animals suffered significant adverse effects.

Characterisation of Biological and Functional Properties of Antibodies to rACP.
i) Enzyme-linked immunosorbent assay (ELISA). Individual murine antisera were reacted in ELISA with both rACP and MC58 OM, as described previously (Christodoulides M (1998), Microbiology 144 (Pt 11), 3027-3037.) Absorbance was measured at 450 nm after 10 min of incubation with enzyme substrate, and the ELISA titre, extrapolated from the linear portion of the serum titration curve, was taken as the reciprocal dilution which gave an increase in absorbance of 0.1 U after 10 min. A two-sample t-test was used to compare differences between mean values for ELISA data, with p values <0.05 considered significant.
ii) Western immunoblotting. Samples containing rACP, OM and whole cell lysate preparations were separated on SDS-PAGE and then transferred to nitrocellulose by semi-dry blotting. After incubation with murine or rabbit sera, immunological reactivity was detected by using anti-mouse/rabbit immunoglobulin-alkaline phosphatase conjugate (Bio-Rad, UK) as described previously (Christodoulides M, (1998) Microbiology 144 (Pt 11), 3027-3037).
iii) Fluorescence Activated Cell Sorting (FACS). An overnight culture of bacteria was collected by centrifugation and cold 70% (v/v) ethanol (2 ml) was added to the pellet, which was then stored at −20° C. for 1 h to permeabilize the capsule. Bacteria were washed twice with sterile PBS containing 1% (w/v) BSA and suspended to 2×108 CFU/ml. Next, bacteria (1 ml) were centrifuged (2,200 g for 3 min), suspended in 200 μl neat rabbit antiserum and incubated at 37° C. for 30 min. After washing with PBS, bacteria were incubated with 100 μl of FITC-conjugated goat anti-rabbit IgG ( 1/50 dilution; Dako, UK) at room temperature for 30 min. Bacteria were fixed with a 0.4% (w/v) para-formaldehyde solution at room temperature for 30 min. Samples were analysed on a FACSAria Flow Cytometer (BD Biosciences, USA).
v) Complement-mediated killing of meningococci. The bactericidal activities of pooled antisera were determined with 5% (v/v) baby rabbit serum (AbD serotec, UK) as a source of exogenous complement, as previously described (Christodoulides (1998) Microbiology 144 (Pt 11), 3027-3037). Murine antisera raised to OM were used as a positive control. Complement-dependent bactericidal activity was determined from the numbers of bacteria surviving in the presence of serum and complement compared to the numbers surviving with complement but without test serum. Sera that showed bactericidal activity (>50%) in two or more dilutions were considered positive.

### Sequencing the Acp Gene of Meningococcal Strains

The acp gene of selected meningococcal strains was sequenced commercially (Geneservice, Oxford, UK) using the primer Seq2095 (5′-cgggatacgccgacattaga-3′ SEQ ID No. 10).

### Constructing an Acp Knockout Mutant

Mutagenesis was achieved by heterologous allelic exchange. Primers KO2095F (5′-cgggctgaaccagatagact-3′ SEQ ID No. 11) and KO2095R (5′-gctccagtttggtacggaga-3′ SEQ ID No. 12) were used to amplify the 2.9 kb DNA segment from the genomic DNA of the ACP-mutant 35/11 while the amplified PCR product from the wild-type MC58 should be 1.3 kb. The ACP-strain 35/11, derived from strain 8013 (serogroup C), was one of the strains in the mutant library generated by random insertion of a mini-transposon (1.6 kb) (Rusniok C, et al. (2009) Genome Biol. 10, R110). The 2.9-kb PCR product was gel-purified, cleaned and used for transformation of MC58. Transformants were screened by PCR and selected MC58ΔACP strain(s) were proved by western blot using rabbit antisera. Sequencing of the 2.9-kb DNA segment amplified from MC58ΔACP showed that the adjacent proteins NMB2094 and NMB2096 were unaffected by the mutagenesis procedure. The transformation protocol of van Dam and Bos (van Dam V, Bos MP (2012) in Neisseria meningitidis Advanced Methods and Protocols, ed. Christodoulides, M. (Humana Press, New York), pp. 55-72) was used for the non-piliated strain MC58¢18 due to the low transformation rate of non-piliated strains. The growth rates of wild-type and ΔACP mutant bacteria were similar (p>0.05).

### Constructing Acp Complemented Strains.

Primers Com2095F (5′-ggctatttaattaaatgaaacactgaccaccgc-3′ SEQ ID No. 13) and Com2095R (5′-ttaacgtggggaacagtctt-3′ SEQ ID No. 14) were used to amplify acp gene from MC58. The sequence ttaattaa represents the restriction site for PacI. The other restriction enzyme used was PmeI. The acp gene was cloned into the pGCC4 vector and proved by sequencing using an upstream primer-LacP (5′-cggttctggcaaatattctg-3′ SEQ ID No. 15). Next, the pGCC4-acp was transformed into MC58ΔACP using the method of Stohl and Seifert (Stohl E A, (2001) Mol. Microbiol. 40, 1301-1310) and the complementary strains were identified by PCR screening.

### Cell Culture

Human Chang conjunctival epithelial cells and Hep2 (epidermoid laryngeal carcinoma) cells (European Type Culture Collection, Porton Down, UK) were cultured in Dulbecco's modified Eagles medium supplemented with Glutamax-1 and sodium pyruvate (DMEM) (Lonza, UK) and 5% (v/v) decomplemented fetal calf serum (dFCS, Lonza). Primary human umbilical vein endothelial cells (HUVECs) were obtained from PromoCell (Heidelberg, Germany) and also from human tissue collected locally by Dr. T. Millar (University of Southampton Faculty of Medicine). HUVECs were grown in DMEM supplemented with 20% (v/v) dFCS and endothelial cell growth supplement (PromoCell). Human meningothelial (M61) and transitional (M53) histological subtype meningioma cells were obtained from surgically removed tumours and characterised cytologically as described previously (Hardy S J, (2000) Mol. Microbiol. 36, 817-829.) Cell lines (passage 5-9) were grown in DMEM with 10% (v/v) dFCS on collagen-coated (type I collagen from rat tail (BD Biosciences), 50 μg/ml in 0.02 M acetic acid) tissue cultureware. All cells were cultured in a humidified atmosphere at 37° C. with 5% (v/v) CO₂.

### Measurement of Total Bacterial Association to Human Cells

Following the method of Virji et al. (Virji M, (1993) Mol. Microbiol. 10, 499-510), cells in triplicate wells of a 24 well tissue culture plate were infected with ˜2-5×10⁷ CFU/ml of bacteria in DMEM containing 1% (v/v) dFCS. After 3 h incubation (37° C. with 5% (v/v) CO₂), the monolayers were washed gently 4 times with PBS and 250 μl of a lysis solution of PBS containing 1% (w/v) saponin (Sigma-Aldrich, UK) and 1% (v/v) dFCS, added to each well. After incubation for 15 min, viable counts of bacteria were made on GC agar plates and the data were analysed by independent t test, with p<0.05 considered significant.

### Measurement of Bacterial Invasion of Human Cells

Cell monolayers were infected with bacteria as described above. After washing with PBS, 1 ml of gentamicin (200 μg/ml) in DMEM containing 1% (v/v) dFCS was added per well and incubated for 90 min to eliminate the extracellular and attached bacteria (Virji M, et al. (1995) Mol. Microbiol. 18, 741-754). The monolayers were washed 4 times and then lysed with saponin lysis solution to release the internalised bacteria. To determine whether any observed invasion was dependent on actin microfilament activity, cell monolayers (n=3) were pre-treated with 1 μg/ml of cytochalasin D (Sigma-Aldrich, UK) for 30 min before addition of bacteria (Virji M, et al. (1995) Mol. Microbiol. 18, 741-754). In parallel, measurement of total association was also determined and the invasion data were shown as the percentage internalisation using the formula: (number of internalised bacteria/number of total associated bacteria)×100%. Differences were compared using the Mann-Whitney U test, with a p value <0.05 considered significant.

### Results

### Nm-ACP Plays a Role in Meningococcal Adhesion and Invasion of Human Cells

The potential role of Nm-ACP in pathogenesis was investigated by infecting human cell cultures in vitro with wild-type MC58 and MC58ΔACP and comparing bacterial association and invasion. Compared with wild-type MC58, there was a significant reduction in association of MC58ΔACP bacteria with all human cell types (FIG. 1A). In rank order, MC58ΔACP showed a ˜75% reduction of mean associated bacterial numbers with both Chang and Hep2 epithelial cells (p<0.05), ˜40% reduction for HUVECs (p<0.05) and ˜20-35% reduction to meningioma cell lines (p<0.05). Furthermore, the MC58ΔACP complemented strain restored the numbers of associated bacteria to all three cell types to levels similar to those of the wild-type strain (p>0.05). To further demonstrate that Nm-ACP played a role in mediating meningococcal association with epithelial cells, rabbit anti-rACP antibody was added to cell cultures during infection with wild-type MC58. Addition of 10% and 1% (v/v) decomplemented rabbit anti-rACP sera decreased the total associated bacterial numbers by ˜85% and 75% (p<0.001), respectively (FIG. 1B).

A potential role for Nm-ACP in cellular invasion was investigated using the standard gentamicin assay. Initially, internalisation of the wild-type capsulated MC58 and MC58ΔACP strains by Chang, Hep2 and HUVECs was examined, but bacterial recovery after gentamicin treatment was very low and not significant between the variants (p>0.05). These data suggested that Nm-ACP did not play a role in invasion of encapsulated strains, so the ability of non-capsulated ACP-expressing and ΔACP strains to invade was compared (FIG. 2). Moreover, to avoid the potentially masking effects of the major meningococcal host binding factors of pili, Opa and Opc proteins, invasion of a non-capsular strain lacking their expression i.e. MC58¢18 (Cap-Pil-Opa-Opc-ACP+) was compared with its Nm-ACP knockout mutant, MC58¢18ΔACP (Cap-Pil-Opa-Opc-ACP−). The percentage internalisation of the Nm-ACP mutant was significantly lower compared to its parent strain in Chang, Hep2 and HUVECs (p<0.05). By contrast to epithelial cells, HUVEC-internalised bacterial numbers were low (<100 CFU/monolayer) in all experiments (n=4); however, the trend of reduced internalisation of the MC58¢18ΔACP compared to MC58¢18 was consistent. As expected, the cytochalasin-treated epithelial and endothelial cells showed significantly reduced numbers of internalised bacteria, by ˜55-98% (p<0.05), suggesting that invasion was likely inhibited by disruption of the actin cytoskeleton. Invasion experiments were not done with meningioma cells, since meningococci show little or no direct invasion of these cells (Hardy S J, (2000) Mol. Microbiol. 36, 817-829).

Cloning, Expression and Purification of rACP

The acp gene was cloned into the pRSETA system and the recombinant plasmids, pRSETA-acp, were transformed into E. coli BL21 (DE3)pLysS for protein expression. The optimal expression time for protein was determined as 4 h after IPTG induction. Analysis of the lysate prepared from E. coli BL21 (DE3)pLysS pRSETA-acp revealed that the recombinant (r)ACP was insoluble and therefore purified under denaturing conditions. The rACP was precipitated from 6M GuHCl using TCA and yielded pure protein, as judged by SDS-PAGE. The Mr of rACP, which has a N-terminal leader sequence of 39 amino acid that contains the 6×His tag, was 17.8 kDa and the LAL assay showed no detectable LPS (<0.125 pg/mg rACP).

Antigenicity of rACP

Pure rACP was used for immunisation studies in adjuvant formulations that are suitable for human immunisation and the murine humoral immune response was studied initially by ELISA against rACP (FIG. 3A). High titres of antibodies that reacted with rACP protein were raised using all the different adjuvant and delivery systems. The rACP adsorbed to Al(OH)₃ induced statistically higher mean titres (133,000) than rACP in saline (22,000), ZW 3-14 micelles (10,000) or in liposomes (8,600) (p<0.05). The addition of MPLA into ZW3-14 micelles and liposomes increased mean antibody titres to 49,000 and 41,000 respectively, but these values did not reach statistical significance compared to mixtures without MPLA (p>0.05). In addition, immunisation with MC58 OM on Al(OH)₃ also induced antibodies that reacted with rACP (FIG. 3A).

The specificity of the immune response against rACP was also investigated by western blotting. Pooled murine antisera raised against rACP in the different formulations ( 1/200 dilution) reacted with both whole-cell lysate and OM preparation of the homologous strain MC58, recognising specifically native ACP with Mr ˜13 kDa.

Additionally, all the antisera raised to formulations containing rACP showed reactivity to native Nm-ACP present in MC58 OM and there were no significant differences between the different adjuvant groups (p>0.05) (FIG. 3B). As expected, significantly higher (p<0.05) anti-OM antibodies were induced by immunisation with OM compared to rACP preparations.

Expression of Nm-ACP on the surface of meningococci was examined by FACS using rabbit antisera raised to rACP. Post-immune rabbit antisera, (with ELISA titres of ˜140-160,000 against rACP and ˜3,000 against MC58 OM) were tested on wild-type (ACP+) MC58 bacteria and showed a significant increase in FITC fluorescence recorded events with a right-shift (FIG. 4A). By contrast, post-immune sera showed no significant reactivity against the MC58ΔACP (ACP−) strain (FIG. 4B).

Nm-ACP is Highly Conserved Amongst Meningococci

The DNA sequencing results of the acp gene in the inventors collection of isolates from colonised individuals and from patients were translated to amino acid sequences and aligned (FIG. 5A). There were 3 types of Nm-ACP identified: type I (strains MC58 and MC168), type II (strains MC54, L2470, MC161, MC162, MC172, MC173, MC174, MC179, MC180 and MENC11) and type III (strain MC90 only). The different Nm-ACP proteins shared a high degree of similarity (98-99%): type II ACP protein had only one amino acid difference compared to type I ACP (Asp25→Asn25), whereas type III ACP had 2 substitutions (Asp25→Asn25 and Ala12→Ser12) when compared to type I ACP.

Using the Basic Local Alignment Search Tool (NCBI website), showed that there were only 2 distinct inferred Nm-ACP protein sequences in meningococcal strains, which were identical to the type I and type II proteins identified in our strain collection. The BIGS Database was also accessed, this database includes the complete genome sequence data for 205 Neisseria strains (31 DNA alleles in total), of which 173 were meningococci (Nm). In this collection, meningococci only possessed genes corresponding to the type I Nm-ACP (encoded by 15 strains represented by allele 1) or to type II Nm-ACP (encoded 158 strains represented by allele 2). Type II Nm-ACP protein was exclusively encoded by meningococci, while a variety of Neisseria strains, including N. sicca (Ns), N. polysaccharea (Np) and N. lactamica (NI), contained the acp gene encoding type I ACP. Notably, genes encoding type I, II and III ACP proteins were not present in N. gonorrhoeae, but gonococci expressed 2 different ACP proteins that showed 95% similarity with meningococcal ACP (FIG. 5A, B).

In order to investigate Nm-ACP expression, bacterial lysates from our strains that encoded type I, II or III proteins were reacted with rabbit anti-rACP sera in western blot. Antisera reacted with a ˜13 kDa band present in all the strains and of similar intensity (FIG. 6). In addition, a lysate of MC58ΔACP was used as a negative control and as expected, showed no reactivity.

rACP Elicits Cross-Strain Bactericidal Antibody

Initially, murine antisera were tested for their ability to promote complement-mediated killing of the homologous meningococcal strain MC58 (Table 1). rACP in liposomes, ZW3-14 micelles or in saline alone induced antisera with the highest bactericidal antibody titres (512).

TABLE 1
Bactericidal activity of pooled antisera raised against
rACP formulations, for the homologous strain MC58.

Serum bactericidal titre*
against MC58

| Formulation | +rACP | −rACP |
|---|---|---|
| Saline | 512 | <4 |
| Al(OH)₃ | 128 (128, 512) | <4 |
| ZW 3-14 | 512 | <4 |
| ZW 3-14 + MPLA | <4 | <4 |
| Liposome | 512 (128, 1024) | <4 |
| Liposome + MPLA | <4 | <4 |

[*The titres are expressed as the reciprocal of the highest dilution at which 50% killing was observed. Titres for normal mouse serum and sera from mice immunised with MC58 OM were <4 and 20,000 respectively. Data are the median values, with the range of values in parenthesis, for SBA from three or more independent measurements of bactericidal activity of all pooled serum samples. Single values denote that the SBA titres were identical from the independent experiments.]

Notably, rACP adsorbed to Al(OH)₃ also induced bactericidal antibodies. However, addition of MPLA to rACP in liposomes and micelles resulted in a complete loss of bactericidal activity. Pooled murine antisera with bactericidal antibodies to type I Nm-ACP (strain MC58) were also tested for bactericidal activity against strains expressing type II and type III Nm-ACP. Antisera to type I Nm-ACP in liposomes, saline or Al(OH)₃ showed identical bactericidal activity for strains MC179 (type II ACP) and MC90 (type III ACP) (Table 2). No bactericidal activity was observed for antisera from sham immunised animals.

TABLE 2
Bactericidal activity of pooled murine anti-
rACP sera for heterologous strains.

| ACP | Serum bactericidal titre* | | |
|---|---|---|---|
| type/ | elicited by rACP in | | |
| strain | Saline | Liposomes | Al(OH)₃ |
| I/MC58 | 512 | 512 | 128 |
| | | (128, 1024) | (128, 512) |
| II/MC179 | 512 | 512 | 64 |
| | (128, 4096) | (128, 2048) | (64, 128) |
| III/MC90 | 512 | 256 | 64 |
| | (128, 4096) | (128, 2048) | (64, 128) |

[*The titres are expressed as the reciprocal of the highest dilution at which 50% killing was observed. Data are the median values, with the range of values in parenthesis, for SBA from three or more independent measurements of bactericidal activity of all pooled serum samples. Single values denote that the SBA titres were identical from the independent experiments.]

### DISCUSSION

Nm-ACP fulfils several criteria for a meningococcal vaccine antigen. The protein was expressed by all strains from a collection of patient and carriage strains and was highly conserved with >98% similarity in protein amino acid sequences. Only two predicted proteins (type I and II) were found in the 205 meningococcal strains in the BIGS database, with one additional protein (type III) expressed by a single strain from our collection. Interestingly, type II Nm-ACP is present exclusively in N. meningitidis, whereas type I Nm-ACP is not only found in meningococci but also in other Neisseria strains colonising the nasopharyngeal mucosal epithelium, including N. sicca, N. polysaccharea and N. lactamica. By contrast, gonococcal ACP was different to meningococcal type I, II and III proteins, suggesting that subtle variation in Neisseria ACP may contribute to directing a bacterial tropism towards colonizing either the nasopharyngeal or urogenital tract mucosal epithelium.

The rACP induced significant complement-mediated SBA against meningococci. Notably, SBA towards the homologous strain induced by rACP in saline was similar to the SBA induced by rACP in liposomes, suggesting that purification of rACP under denaturing conditions did not adversely influence functional immunogenicity. Moreover, antisera to type I ACP showed cross-protection by killing heterologous strains expressing type II and III ACP at similar SBA levels. The nature of both antigen and adjuvant are both critical to the quality of the SBA response: thus, for rACP, adsorption to Al(OH)₃ reduced bactericidal titres and introduction of exogenous MPLA abolished SBA. These observations have been reported for other meningococcal antigens and highlight the need for careful matching of adjuvant to antigen.

By comparing the interactions of wild-type (ACP+), mutant (ACP−) and complemented strains, Nm-ACP was identified as a new adhesin showing cell tropism, whereby adherence was greatest for epithelial cells, then endothelial cells and finally meningeal cells. Nm-ACP mediated adhesion of capsulated meningococci and the tropism is likely to be a consequence of the relative expression levels of as yet uncharacterised recognition receptors for Nm-ACP on these different host cell types. Nm-ACP is also an invasin that facilitated bacterial internalisation into human epithelial and endothelial cells. The acp gene is present in all meningococcal strains studied and the amino acid sequences were more highly conserved.

1-20. (canceled)

21. An immunogenic composition comprising a Nm-ACP protein or an immunogenic part thereof or an analogue thereof, wherein the composition is capable of eliciting an immune response when administered to a human or non-human animal.

22. The immunogenic composition of claim 21, wherein the immune response elicited is a protective immune response.

23. The immunogenic composition of claim 22, wherein the immune response affects the ability of N. meningitidis to infect an immunised animal.

24. The immunogenic composition of claim 22, wherein the immune response prevents a N. meningitides and/or a N. gonorrhoeae infection.

25. The immunogenic composition of claim 21, wherein the immune response is against one or more of the group consisting of: N. meningitidis serogroup B strains, N. meningitidis serogroup A strains, N. meningitidis serogroup C strains, N. meningitidis serogroup W135 strains, and N. meningitidis serogroup Y strains.

26. The immunogenic composition of claim 21, wherein the Nm-ACP protein is a recombinant protein.

27. The immunogenic composition of claim 21, wherein the Nm-ACP protein is encoded by the sequence selected from the group consisting of: SEQ ID NOs: 1, 2, 3, 4, 5, and 7.

28. The immunogenic composition of claim 21, further comprising at least one of an antigen or an adjuvant.

29. The immunogenic composition of claim 28, wherein the antigen is a N. meningitidis antigen and wherein the adjuvant is monophosphoryl Lipid A.

30. A pharmaceutical composition comprising a Nm-ACP protein or an immunogenic part thereof or an analogue thereof and a pharmaceutically acceptable carrier or excipient.

31. The pharmaceutical composition of claim 30, wherein the Nm-ACP protein or an immunogenic part thereof or an analogue thereof is incorporated into liposomes or detergent micelles.

32. The pharmaceutical composition of claim 30, further comprising at least one of an adjuvant or an antigen.

33. The pharmaceutical composition of claim 32, wherein the antigen is a N. meningitidis antigen and wherein the adjuvant is monophosphoryl Lipid A.

34. A method of eliciting an immune response in a human or a non-human animal comprising administering a composition comprising immunologically effective amount of a Nm-ACP protein or an immunogenic part thereof or an analogue thereof.

35. The method of claim 34, wherein the composition further comprises at least one of an adjuvant or an antigen.

36. The method of claim 34, wherein the Nm-ACP protein is a recombinant protein.

37. The method of claim 34, wherein the Nm-ACP protein is encoded by the sequence selected from the group consisting of: SEQ ID NOs: 1, 2, 3, 4, 5, and 7.

38. The method of claim 34, wherein the immune response is against N. meningitidis and/or N. gonorrhoeae.

39. The method of claim 34, wherein the immune response is one or more of the group consisting of: N. meningitidis serogroup B strains, N. meningitidis serogroup A strains, N. meningitidis serogroup C strains, N. meningitidis serogroup W135 strains, and N. meningitidis serogroup Y strains.

40. The method of claim 34, wherein immune response protects a human or non-human animal from the effects of infection by N. meningitidis and/or N. gonorrhoeae.


SEQUENCE LISTING (15 sequences)

SEQ ID NO: 1 - protein, 124 aa, Neisseria meningitidis
MKLLTTAILSSAIALSSMAAAAGTDNPTVAKKTVSYVCQQGKKVKVTYGFNKQGLTTYASAVINGKRVQMPVNLDKSDNVETFYGKEGGYVLGTGVMDGKSYRKQPIMITAPDNQIVFKDCSPR

SEQ ID NO: 2 - protein, 124 aa, Neisseria meningitidis
MKLLTTAILSSAIALSSMAAAAGTNNPTVAKKTVSYVCQQGKKVKVTYGFNKQGLTTYASAVINGKRVQMPVNLDKSDNVETFYGKEGGYVLGTGVMDGKSYRKQPIMITAPDNQIVFKDCSPR

SEQ ID NO: 3 - protein, 124 aa, Neisseria meningitidis
MKLLTTAILSSAISLSSMAAAAGTNNPTVAKKTVSYVCQQGKKVKVTYGFNKQGLTTYASAVINGKRVQMPVNLDKSDNVETFYGKEGGYVLGTGVMDGKSYRKQPIMITAPDNQIVFKDCSPR

SEQ ID NO: 4 - protein, 124 aa, Neisseria gonorrhoeae
MKLLTTAILSSAIALSSMAAAAGTNNPTVAKKTVSYVCQQGKKVKVTYGFNKQGLTTYASAVINGKRVQMPINLDKSDNMDTFYGKEGGYVLSTGAMDSKSYRKQPIMITAPDNQIVFKDCSPR

SEQ ID NO: 5 - protein, 123 aa, Neisseria gonorrhoeae
MKLLTTAILSSAIALSSMAAAGTDNPTVAKKTVSYVCQQGKKVKVTYGFNKQGLTTYASAVINGKRVQMPINLDKSDNMDTFYGKEGGYVLSTGAMDSKSYRKQPIMITAPDNQIVFKDCSPR

SEQ ID NO: 6 - DNA, 375 bp, Neisseria meningitidis
atgaaacttctgaccaccgcaatcctgtcttccgcaatcgcgctcagcagtatggctgccgccgctggcacggacaaccccactgttgcaaaaaaaaccgtcagctacgtctgccagcaaggtaaaaaagtcaaagtaacctacggcttcaacaaacagggtctgaccacatacgcttccgccgtcatcaacggcaaacgcgtgcaaatgcctgtcaatttggacaaatccgacaatgtggaaacattctacggcaaagaaggcggttatgttttgggtaccggcgtgatggatggcaaatcctaccgcaaacagcccattatgattaccgcacctgacaaccaaatcgtcttcaaagactgttccccacgttaa

SEQ ID NO: 7 - DNA, 558 bp, Neisseria meningitidis
aattttgtttaactttaagaaggagatatacatatgcggggttctcatcatcatcatcatcatggtatggctagcatgactggtggacagcaaatgggtcgggatctgtacgacgatgacgataaggatcgatggggatccgagctcgagatgaaacttctgaccaccgcaatcctgtcttccgcaatcgcgctcagcagtatggctgccgccgctggcacggacaaccccactgttgcaaaaaaaaccgtcagctacgtctgccagcaaggtaaaaaagtcaaagtaacctacggcttcaacaaacagggtctgaccacatacgcttccgccgtcatcaacggcaaacgcgtgcaaatgcctgtcaatttggacaaatccgacaatgtggaaacattctacggcaaagaaggcggttatgttttgggtaccggcgtgatggatggcaaatcctaccgcaaacagcccattatgattaccgcacctgacaaccaaatcgtcttcaaagactgttccccacgttaatagaagcttgatccggctgctaacaaagcccga

SEQ ID NO: 8 - DNA, 32 bp, Artificial Sequence
  forward primer NMB2095F
ggctatctcgagatgaaacttctgaccaccgc

SEQ ID NO: 9 - DNA, 35 bp, Artificial Sequence
  Reverse primer
ggctataagcttctattaacgtggggaacagtctt

SEQ ID NO: 10 - DNA, 20 bp, Artificial Sequence
  primer Seq 2095
cgggatacgccgacattaga

SEQ ID NO: 11 - DNA, 20 bp, Artificial Sequence
  Primer KO2095F
cgggctgaaccagatagact

SEQ ID NO: 12 - DNA, 20 bp, Artificial Sequence
  Primer KO2095R
gctccagtttggtacggaga

SEQ ID NO: 13 - DNA, 34 bp, Artificial Sequence
  Primer Com2095F
ggctatttaattaaatgaaacttctgaccaccgc

SEQ ID NO: 14 - DNA, 20 bp, Artificial Sequence
  Primer Com2095R
ttaacgtggggaacagtctt

SEQ ID NO: 15 - DNA, 20 bp, Artificial Sequence
  Primer lacP
cggttctggcaaatattctg